(19) United States
(12) Patent Application Publication
(10) Pub. No.: US 2008/0033208 A1
(43) Pub. Date: Feb. 7, 2008

(54) Process for Manufacturing Optically Pure (R) or (S)-5-(2-Aminopropyl)-2-Methoxybenzene Sulfonamide

(76) Inventors: Dilipkumar Chandubhai Ramolia, Gujarat (IN); Dnyaneshwar Sitaram Patil, Gujarat (IN); Dharmeshkumar Arvindbhai Patel, Gujarat (IN); Rajivkumar Sharma, Gujarat (IN); Virendra Kumar Agarwal, Gujarat (IN)

Correspondence Address: LADAS & PARRY, 26 WEST 61ST STREET, NEW YORK, NY, 10023, US

(21) Appl. No.: 10/589,999

(22) PCT Filed: Feb. 22, 2005
(86) PCT No.: PCT/IN05/00055

§ 371 Date: Jul. 2, 2007

(30) Foreign Application Priority Data
Feb. 23, 2004 (IN) 218/MUM/2004

(51) Int. Cl. C07C 311/16

(52) U.S. Cl. 564/86

(57) ABSTRACT

The invention relates to an improved process for the manufacture of enantiomerically pure R-(+) or S-(+)-5-(2-amino-propyl)-2-methoxybenzene sulfonamide by resolution of (R,S)-5-(2-aminopropyl)-2-methoxybenzene sulfonamide with D-(−) or L-(+)-tartaric acid to form a mixture of diastereomeric salts, separating the diastereomeric salts by kinetic resolution in a mixture of solvent systems of the kind such as herein described, in the specified time and temperature range to provide said R-(−)-5-(2-aminopropyl)-2-methoxybenzene sulfonamide or S-(+)-5-(2-aminopropyl)-2-methoxybenzenesulfonamide with excellent chiral purity more than 99.9%.

### FIELD OF THE INVENTION

The present invention relates to an improved and simplified process for the preparation of enantiomerically pure R-(−) or S-(+)-5-(2-aminopropyl)-2-methoxybenzenesulfonamide by resolution of (R,S)-5-(2-aminopropyl)-2-methoxybenzenesulfonamide with D-(−) or L-(+)-tartaric acid, respectively, which is described in Indian Patent Application No.: 1153/MUM/2002; PCT Patent Application No.: PCT/IN02/00244;filed on 26 Dec. 2002, filed by Cadila Healthcare Ltd. In particular, the present invention relates to an improved process for the manufacturing of a highly optically pure (R)-(−)-5-(2-aminopropyl)-2-methoxybenzene sulfonamide (Chiral purity: >99.9%) of Formula I, which is a key intermediate for the preparation of Tamsulosin having Formula II (EP-34432, U.S. Pat. No. 4,703,063) useful in the treatment of patients with symptomatic Benign Prostatic Hyperplasia (BPH).

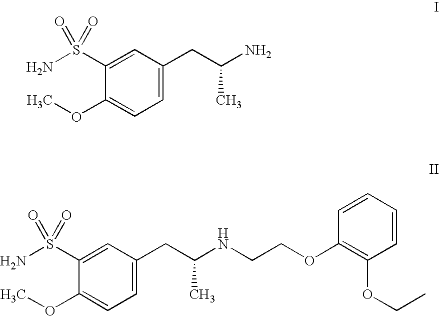

### BACKGROUND OF THE INVENTION

Tamsulosin is a selective blocker of α_{1c}-receptors, which shows a selective effect during treatment of problems related to hyperplasic prostate without influencing blood pressure or heart action (Honda K. and Nakagawa C.: α₁-adrenoceptor antagonist effect of optical isomer YM-12617 in rabbit lower urinary tract and prostate-J. Pharma. Exp. Ther. 239,512 (1986)).

The group of compounds as described in patent EP 34 432 have characteristic for their ability to block α-adrenergic receptors, which led to their use in treating a number of diseases, especially hypertension, congestive heart failure or problems related to the urinary tract.

This leads to the effort to effectively synthesize the optically active R-(−)-5-(2-aminopropyl)-2-methoxy benzenesulfonamide, which is the key intermediate of Tamsulosin.

PCT International Application No.: PCT/IN02/00244 filed on 26 Dec. 2002 presented a synthesis of R-(−)-5-(2-aminopropyl)-2-methoxy benzenesulfonamide from the reduction of 5-(2-Hydroxyiminopropyl)-2-methoxy benzenesulfonamide followed by optical resolution of (R,S)-5-(2-amino propyl)-2-methoxy benzenesulfonamide with D-(−)-tartaric acid.

### OBJECTS OF THE PRESENT INVENTION

In continuation of our work, we have adopted the kinetic resolution technique to resolve (R,S)-5-(2-amino-propyl)-2-methoxy benzenesulfonamide into R-(−)-5-(2-aminopropyl)-2-methoxy benzenesulfonamide and S-(+)-5-(2-aminopropyl)-2-methoxy benzenesulfonamide using D-(−) or L-(+)-tartaric acid as cheap resolving agent. Thus it is the most commercially viable process.

Accordingly, it is an object of the present invention to provide a plant friendly and commercially viable process for the manufacture of R-(−)-5-(2-aminopropyl)-2-methoxybenzenesulfonamide (I). This objective of the present invention can only be achieved, if one can invent a suitable diastereomeric salt of (R,S)-5-(2-aminopropyl)-2-methoxybenzenesulfonamide, whose differential solubility properties can be exploited in a suitable solvent system at an appropriate temperature range to obtain desired optically pure (R)-(−)-5-(2-aminopropyl)-2-methoxybenzenesulfonamide (I) in minimum possible operations.

### DETAILED DESCRIPTION

Thus, the present invention provides a simplified process for the manufacture of optically pure R-(−) or S-(+)-5-(2-aminopropyl)-2-methoxy benzene sulfonamide (I), which comprises the resolution of (R,S)-5-(2-amino propyl)-2-methoxybenzenesulfonamide with D-(−) or L-(+)-tartaric to form a mixture of diastereomeric salts, separating the diastereomeric salts in a mixture of solvent systems of the kind such as described herein at a specified temperature range. The diastereomeric salt thus obtained is kinetically resolved two times in a same solvent system and under similar operational conditions to get desired optical purity (>99.9%). The purified diastereomeric salt is then basified to generate free (R)-(−)-5-(2-amino-propyl)-2-methoxybenzenesulfonamide (I) (Scheme-1).

(R,S)-5-(2-aminopropyl)-2-methoxybenzenesulfonamide can be prepared by the process as disclosed in our previous patent i.e., PCT International Application No.: PCT/IN02/00244 filed on 26 Dec. 2002.

The molar ratio of (R,S)-5-(2-aminopropyl)-2-methoxybenzenesulfonamide to the D-(−)-tartaric acid is 1:1 to 1:1.5, preferably, 1:1.1. The diastereomeric salt formation as well as resolution is preferably carried out in a same solvent or the mixture of solvent system.

It has been observed that the resolution of (R,S)-5-(2-aminopropyl)-2-methoxybenzenesulfonamide with D-(−)-tartaric acid is largely governed by the polarity of the solvent system used. The solvent system preferred is a combination of alcoholic solvents such as methanol, ethanol, 1-propanol and 2-propanol with 0-80% (v/v) of dipolar solvents such as N,N-dimethylformamide, N,N,-dimethylacetamide, N-methyl-2-pyrrolidone; dimethylsulfoxide or water. Though water alone can also be used for salt formation as well as resolution at ambient temperature, however in order to obtain optimum yield and optical purity, it is also used in combination with alcoholic solvents.

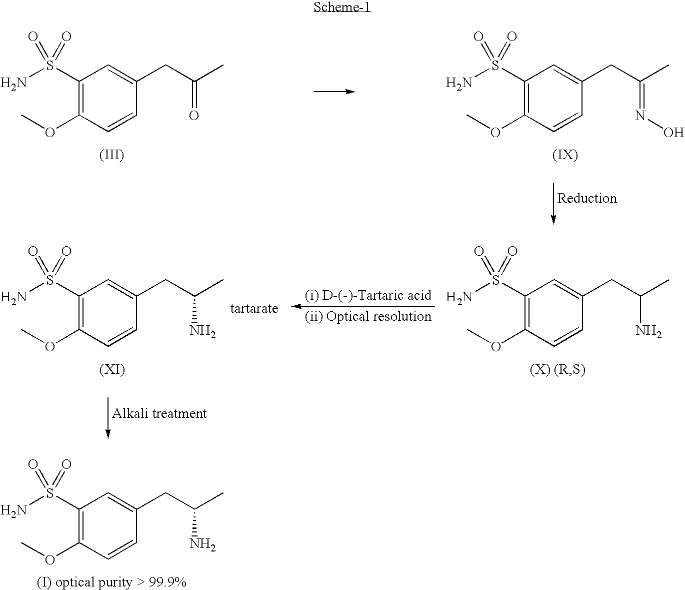

The temperature also plays a key role in kinetic resolution for obtaining the optically pure (R)-(−)-5-(2-aminopropyl)-2-methoxybenzenesulfonamide. It has been also observed that if the resolution is carried out at 10-75° C. temperature, the desired product is obtained in a higher optical purity. Thus the temperature between 30-65° C. range provides the best results.

The reaction time may also vary between 0 to 26 hours after the addition of the amine to the tartaric acid; however under optimal reaction conditions, the preferred reaction time is 4-8 hours to obtain the optimum yield with desired optical purity.

The separated diastereomeric salt (R:2S,3S) from the reaction mass is isolated by filtration. Moderately resolved diastereomeric salt may also be further purified twice using same solvent system, under similar operational conditions, the purified salt is then treated with a base, i.e. alkali hydroxides, carbonates and hydrogen carbonates, preferably sodium hydroxide to bring pH 9.5-10.0 to obtain (R)-(−)-5-(2-aminopropyl)-2-methoxybenzenesulfonamide as free base.

The mother liquor obtained from I and II crystallization contains 70-85% R-isomer of 5-(2-aminopropyl)-2-methoxybenzenesulfonamide as tartarate salt, that can be mixed in another batch during I purification to enhance the productivity.

The resolving agent D-(−)-tartaric acid can be recovered from the aqueous mother liquor by usual known methods, as reported in literature and reused for the same resolution.

Besides D-(−)-tartaric acid, L-(+)-tartaric acid may also be used as a resolving agent. The resolution of (R,S)-5-(2-aminopropyl)-2-methoxybenzenesulfonamde using L-(+)-tartaric acid is also carried out in the same fashion. However in this case, (S:2R,3R) diastereomeric salt separates out from the reaction mixture which after desalting gives the S-(+)-5-(2-aminopropyl)-2-methoxy benzene sulfonamide.

In a preferred embodiment, (R,S)-5-(2-aminopropyl)-2-methoxybenzene sulfonamide is treated with 1.1 molar ratio of D-(−)-tartaric acid in 8.5 volumes of methanol and 1.7 volumes of dimethylformamide at 60-65° C. for 6 hours. The obtained solid is filtered at 60-65° C. The cake is slurrified with 3.0 volume of methanol, filtered and washed with 0.25 volumes of methanol. The wet product thus obtained is dried at 50-55° C. till constant weight to give 70-75% yield of diastereomeric salt containing R-isomer 84-87% and S-isomer 13-16% as determined on chiral HPLC.

The obtained salt is refluxed in 6.5 volumes of aqueous methanol containing 38.5% (v/v) water for 1 hour, then cooled to 40-45° C. and maintained the same temperature for another 2 hours. The resulting mass is further cooled to 30-35° C. and aged for 6 hours at same temperature range. The obtained solid is filtered and washed twice with 0.25 volumes of methanol as that of tartarate salt. The salt is dried at 50-55° C. till constant weight to give 65-70% yield of purified diastereomeric salt containing R-isomer 98-99% and S-isomer 1-2% as determined on chiral BPLC.

The above obtained purified salt is again refluxed in 6.5 volumes of aqueous methanol containing 38.5% (v/v) water for 1 hour, then cooled to 40-45° C. and maintained this temperature for 2 hours. The resulting mass is further cooled to 30-35° C. and aged for 6 hours at same temperature range. The solid is filtered and then washed twice with 0.25 volumes of methanol as that of the tartarate salt. The salt is dried at 50-55° C. till constant weight to give 65-70% yield of diastereomeric salt containing R-isomer 99.90-99.95% and S-isomer 0.05-0.10% as determined on chiral HPLC.

The present invention describes a simple methodology to get first moderately resolved tartarate salt of (R,S)-5-(2-aminopropyl)-2-methoxybenzene sulfonamide, which on subsequent two simple purification with same solvent system provides a diastereomeric salt with high optical purity i.e., more than 99.9%.

The present invention provides a highly optical pure (>99.9%) 5-(2-aminopropyl)-2-methoxybenzenesulfonamide with overall yield of 33-35% (without recyclable crop) from racemic 5-(2-aminopropyl)-2-methoxybenzene sulfonamide. The present method is also capable of reducing S-isomer from 50% to less than 0.05% thereby increasing the optical purity of R-(−)-5-(2-aminopropyl)-2-methoxybenzenesulfonamide from 50.0% to 99.95% exhibiting a successful kinetic resolution of diastereoisomeric salt.

The present invention is further described in greater detail as illustrated in the following of examples.

### Resolution of (R,S)-(−)-5-(2-aminopropyl)-2-methoxybenzenesulfonamide

### EXAMPLE 1

### Resolution of (R,S)-(−)-5-(2-aminopropyl)-2-methoxybenzenesulfonamide

300.0 g (R,S)-5-(2-aminopropyl)-2-methoxybenzenesulfonamide was added in 3000 ml of methanol:water mixture (95:5% v/v) and then heated to 60-65° C. for complete dissolution. 202.9 g of D-(−)-tartaric acid was slowly added at 60-65° C. in the reaction mixture and then maintained 60-65° C. temperature for 6 hours. The crystals were collected by filtration at same temperature (60-65° C.), washed with 2×75 ml methanol and dried at 65-75° C. temperature till constant weight to provide 187.3 g of R-(−)-5-(2-aminopropyl)-2-methoxybenzene sulfonamide tartarate.

Yield: 77.46%

Melting point: 194-195° C. (dec.)

$$[ α ] {25}_{D} - {19.4}^{∘} ( c = 1.0 , {H}_{2} O )$$

### EXAMPLE 2

### Resolution of (R,S)-(−)-5-(2-aminopropyl)-2-methoxybenzenesulfonamide

250 g of (R,S)-5-(2-aminopropyl)-2-methoxybenzenesulfonamide was added in 2550 ml of methanol:dimethyl formamide mixture (80:20% v/v) then heated to 60-65° C. for complete dissolution. 169 g of D-(−)-tartaric acid was slowly added at 60-65° C. in the reaction mixture and then maintained 60-65° C. temperature for 6 hours. The crystals were filtered off. The wet product was taken in 750 ml of methanol and stirred for half an hour, at ambient temperature, was filtered off and washed with 2×62.5 ml methanol, thereby affording 193.3 g. of R-(−)-5-(2-aminopropyl)-2-methoxybenzenesulfonamide tartarate.

Yield: 95.92%

Melting point: 193-194° C. (dec.)

$$[ α ] {25}_{D} - {19.16}^{∘} ( c = 1.0 , {H}_{2} O )$$

### EXAMPLE 3

### Resolution of (R,S)-(−)-5-(2-aminopropyl)-2-methoxybenzenesulfonamide

75.5 kg of (R,S)-5-(2-aminopropyl)-2-methoxybenzenesulfonamide was added in 770.1 L of methanol:dimethyl formamide mixture (8.5:1.7% v/v) then heated to 60-65° C. for complete dissolution. 51.0 kg of D-(−)-tartaric acid was slowly added at 60-65° C. in the reaction mixture and then maintained 60-65° C. temperature for 6 hours. The crystals were filtered off. The wet product was taken in 226.5 L of methanol and stirred for half an hour, at ambient temperature, was filtered off and washed with 2×18.87 L methanol, thereby affording 45.0 kg of R-(−)-5-(2-aminopropyl)-2-methoxybenzenesulfonamide tartarate.

Yield: 95.92%

Melting point: 193-194° C. (dec.)

$$[ α ] {25}_{D} - {19.16}^{∘} ( c = 1.0 , {H}_{2} O )$$

1^{st} Purification of R-(−)-5-(2-aminopropyl)-2-methoxybenzene sulfonamide tartarate

### EXAMPLE 4

[A] A mixture of 100 g of R-(−)-5-(2-aminopropyl)-2-methoxybenzene 5 sulfonamide tartarate (obtained in Example-2), 400 ml methanol and 250 ml water was refluxed for 1 hour. The clear solution was slowly cooled to 40-45° C. and maintained for 2 hours at 40-45° C. The reaction mass further cooled to 28-30° C. and stirred for 6 hours at same temperature. The product was filtered at same temperature and washed with 2×25 ml methanol, dried the solid at 50-55° C. till constant weight to give 63.3 g of R-(−)-5-(2-aminopropyl)-2-methoxy benzene sulfonamide tartarate. [ee:98.38%].

[B] A mixture of 20 g of R-(−)-5-(2-aminopropyl)-2-methoxybenzenesulfonamide tartarate (obtained in Example-2), 160 ml methanol and 60 ml water was refluxed for 1 hour. The clear solution was slowly cooled to 40-45° C. and maintained at 40-45° C. for 2 hours. The reaction mass further cooled to 28-30° C. and stirred for 6 hours at same temperature The product was filtered at same temperature and washed with 2×5 ml methanol, dried the solid at 50-55° C. till constant weight to give 11.1 g of R-(−)-5-(2-aminopropyl)-2-methoxybenzenesulfonamide tartarate. [ee:98.02%].

[C] A mixture of 97.0 g of R-(−)-5-(2-aminopropyl)-2-methoxybenzene sulfonamide tartarate (obtained in Example-2), 388 ml methanol and 170 ml water was refluxed for 1 hour. The clear solution was slowly cooled to 45-48° C. and maintained at 45-48° C. for 2 hours. The reaction mass further cooled to 28-30° C. and stirred for 6 hours at same temperature. The product was filtered at same temperature and washed with 2×24 ml methanol, dried the solid at 50-55° C. till constant weight to give 67.7 g of R-(−)-5-(2-aminopropyl)-2-methoxybenzene sulfonamide tartarate. [ee:96.89%].

[D] A mixture of 101.0 g of R-(−)-5-(2-aminopropyl)-2-methoxybenzene sulfonamide tartarate (obtained in Example-2), 404 ml methanol and 202 ml water was refluxed for 1 hour. The clear solution was cooled to 45-47° C. and maintained at 45-47° C. for 2 hours. The reaction mass further cooled to 28-30° C. and stirred for 6 hours at same temperature. The product was filtered at same temperature and washed with 2×25 ml methanol, dried the solid at 50-55° C. till constant weight to give 64.0 g of R-(−)-5-(2-aminopropyl)-2-methoxybenzene sulfonamide tartarate having ee=97-97.5%.

[E] A mixture of 45.0 kg of R-(−)-5-(2-aminopropyl)-2-methoxybenzene sulfonamide tartarate (obtained in Example-3), 180 L methanol and 90.0 L water was refluxed for 1 hour. The clear solution was cooled to 45-47° C. and maintained at 45-47° C. for 2 hours. The reaction mass further cooled to 28-30° C. and stirred for 6 hours at same temperature. The product was filtered at same temperature and washed with 2×11.25 L methanol, dried the solid at 50-55° C. till constant weight to give 27.1 kg of R-(−)-5-(2-aminopropyl)-2-methoxybenzene sulfonamide tartarate having ee=97-97.5%.

### EXAMPLE 5

### II Purification of R-(−)-5-(2-aminopropyl)-2-methoxybenzenesulfonamide tartarate

[A] A mixture of 56.0 g of R-(−)-5-(2-aminopropyl)-2-methoxybenzene sulfonamide tartarate (obtained in Example-4A), 224 ml methanol and 140 ml water was refluxed for 1 hour. The clear solution was slowly cooled to 40-45° C. and maintained at 40-45° C. for 2 hours. The reaction mass further cooled to 28-30° C. and stirred for 6 hours at same temperature. The product was filtered at same temperature and washed with 2×14 ml methanol, dried the solid at 50-55° C. till constant weight to give 40.7 g of R-(−)-5-(2-aminopropyl)-2-methoxybenzene sulfonamide tartarate having ee=99.98-100.0%.

[B] A mixture of 63.0 g of R-(−)-5-(2-aminopropyl)-2-methoxybenzene sulfonamide tartarate (obtained in Example-4C), 252 ml methanol and 110.2 ml water was refluxed for 1 hour. The clear solution was slowly cooled to 50-55° C. and maintained at 50-55° C. for 2 hours. The reaction mass further cooled to 28-30° C. and stirred for 6 hours at same temperature. The product was filtered at same temperature and washed with 2×15 ml methanol, dried the solid at 50-55° C. till constant weight to give 49.6 g of R-(−)-5-(2-aminopropyl)-2-methoxybenzene sulfonamide tartarate having ee=99.40-99.70%.

[C] A mixture of 59.0 g of R-(−)-5-(2-aminopropyl)-2-methoxybenzene sulfonamide tartarate (obtained in Example-4D), 236 ml methanol and 118 ml water was refluxed for 1 hour. The clear solution was slowly cooled to 50-55° C. and maintained at 50-55° C. for 2 hours. The reaction mass further cooled to 28-30° C. and stirred for 6 hours at same temperature. The product was filtered at same temperature and washed with 2×14 ml methanol, dried the solid at 50-55° C. till constant weight to give 44.7 g of R-(−)-5-(2-aminopropyl)-2-methoxybenzene sulfonamide tartarate having ee=99.40-99.60%.

[D] A mixture of 27.1 kg of R-(−)-5-(2-aminopropyl)-2-methoxybenzene sulfonamide tartarate (obtained in Example-4E), 108.4 L methanol and 54.2 L water was refluxed for 1 hour. The clear solution was slowly cooled to 50-55° C. and maintained at 50-55° C. for 2 hours. The reaction mass further cooled to 28-30° C. and stirred for 6 hours at same temperature. The product was filtered at same temperature and washed with 2×6.77 L methanol, dried the solid at 50-55° C. till constant weight to give 19.6 kg of R-(−)-5-(2-aminopropyl)-2-methoxybenzene sulfonamide tartarate having ee=99.40-99.60%.

### EXAMPLE 6

### Preparation of R-(−)-5-(2-aminopropyl)-2-methoxybenzenesulfonamide

To 10 g tartarate salt (obtained in Example-5A) of R-(−)-5-(2-aminopropyl)-2-methoxybenzene sulfonamide in 10 ml water was added 40% aq. sodium hydroxide solution, to adjust pH between 9.5 -10.0. The reaction mixture was stirred for 1 hour at 25-30° C. temperature. The product obtained was filtered and washed with 2.5 ml water. Dried the product at 55-60° C. till constant weight. 6.0 g of R-(−)-5-(2-aminopropyl)-2-methoxybenzenesulfonamide was collected. Chiral purity: >99.9%

Melting point: 166° C.-167° C.

$$[ α ] { 23}_{D} - {17.31}^{∘} ( c = 1.07 , Methanol )$$

1. A process for the manufacture of highly optical pure R-(−) or S-(+)-5-(2-aminopropyl)-2-methoxybenzenesulfonamide which comprises:
(a) resolving (R,S)-5-(2-aminopropyl)-2-methoxybenzenesulfonamide with D-(−)- or L-(+)-tartaric acid to form a mixture of diastereomeric salts
(b) separating the diastereomeric salt by filtration at desired temperature range
(c) subjecting diastereomeric salt kinetic resolution in a solvent system of the kind such as hereinbefore described;
(d) liberating of optically pure R-(−) or S-(+)-5-(2-aminopropyl)-2-methoxy benzenesulfonamide with optical purity more than 99.9%.

2. A process as claimed in claim 1, wherein said D-(−)- or L-(+)-tartaric acid is employed in 1-1.5 molar ratio with that of (R,S)-5-(2-aminopropyl)-2-methoxybenzenesulfonamide.

3. A process as claimed in claim 2 wherein D-(−)- or L-(+)-tartaric acid is preferably employed in 1.1 molar ratio with that of (R,S)-5-(2-aminopropyl)-2-methoxybenzenesulfonamide.

4. A process as claimed in claim 1, wherein in step (a) protic solvents containing C₁-C₆ are employed for resolution alone or along with varying proportions of dipolar solvents like N,N-dimethylformamide, N,N-dimethylacetamide, dimethyl sulfoxide, N-methyl-2-pyrrilodone and water.

5. A process as claimed in claim 4, wherein said protic solvents are selected from methanol, ethanol, 1-propanol and 2-propanol.

6. A process as claimed in claim 5, wherein ratio of the dipolar solvent to alcoholic solvent varies from 5-20% (v/v).

7. A process as claimed in claim 6, wherein amount of a single solvent or a solvents combination employed as that of (R,S)-5-(2-aminopropyl)-2-methoxybenzenesulfonamide is 5-25 volume.

8. A process as claimed in claim 7, wherein the most preferable amount of methanol and N,N-dimethylformamide employed as that of (R,S)-5-(2-aminopropyl)-2-methoxybenzenesulfonamide is 8.5 and 1.7 volumes respectively.

9. A process as claimed in claim 8, wherein resolution is carried out at a temperature in the range of 20-70° C., preferably, 60-65° C.

10. A process as claimed in claim 9, wherein the reaction mixture is stirred for 0-26 hours, preferably, 6 hours.

11. A process as claimed in claim 1, wherein in step (a), the diastereomeric salt contains the (R) isomer in an amount of from 60-96% and (S) isomer in an amount of from 40-4%, preferably, 80-90% and 20-10% respectively.

12. A process as claimed in claim 1, wherein in step (b), said diastereomeric salt is separated by filtration over nutsche filter or by centrifugation at temperature 30-70° C.

13. A process as claimed in claim 12, wherein the filtration or centrifugation is carried out preferably at a temperature in the range of 60-65° C.

14. A process as claimed in claim 1, wherein I step (b) said kinetic resolution of moderately resolved diastereomeric salt is accomplished in 5-15 volumes of a single solvent or a solvent mixture containing primary, secondary or tertiary alcohol (C₁-C₆) with water to get desired optical purity.

15. A process as claimed in claim 14, wherein said kinetic resolution of moderately resolved diastereomeric salt is accomplished in solvent mixture of 0-10 volumes, preferably, 4 volumes of methanol and 0-5 volumes, preferably 2.5 volumes of water to get desired optical purity.

16. A process as claimed in claim 1, wherein in step (c) the reaction mixture is refluxed for 0.5-4 hours, preferably 1 hour.

17. A process as claimed in claim 1, wherein in step (c) the clear solution is cooled from reflux temperature (65-100° C.) to 30-60° C., in first cooling, preferably 40-55° C. to get optimum results.

18. A process as claimed in claim 24, wherein the temperature of reaction mixture is maintained for 0-10 hours, preferably 2 hours.

19. A process is claimed in claim 18, wherein the reaction mixture is further cooled to 10-40° C., preferably, 30-35° C. in second cooling.

20. A process as claimed in claim 18, wherein the reaction mixture is aged for 0-12 hours preferably 6 hours, before filtration.

21. A process as claimed in claim 20, wherein the diastereomeric salt contains R-isomer in an amount of 93-99%, preferably 98-99% and S-isomer 1-7%, preferably, 1-2%.

22. A process as claimed in claim 1, wherein in step (c) kinetically resolved diastereomeric salt is subjected to 1-2 more purification steps.

23. A process as claimed in claim 22, wherein the optical purity of R-(−)-isomer of 5-(2-aminopropyl)-2-methoxybenzenesulfonamide is enhanced from 98 to 99.95% by methanol: water purification.

24. A process as claimed in claim 1, wherein in step (d) pure diastereomeric salts having optical purity >99.5% is treated with aqueous solution of base i. e. alkali metal hydroxides, carbonates and bicarbonates to get free R-(−) or S-(+)-5-(2-aminopropyl)-2-methoxybenzenesulfonamide with optical purity between 99.5-99.9%, but most preferably aqueous sodium hydroxide solution is used.

25. A process as claimed in claim 1, wherein the mother liquor obtained from I and II purification contains diastereomeric salts, having optical purity 70-85% of R-(−) or S-(+)-5-(2-aminopropyl)-2-methoxy benzenesulfonamide, that can be mixed in another batch during I purification to enhance the productivity.
